SEQUENCE LISTING (14 sequences)

SEQ ID NO: 1 - DNA, 442 bp, Artificial Sequence
  Tag
tgaagtagctccataatcagaagatgcgctaccgtacacaacggcggtttaaactttgtagacctcacgcggctgctcaggtcttgcgcatcccatacttcacatggacggaaagctgtgggtttgtatcgataagccgtaggttcctaggatcgagtcacccaaacaacttcgagagctcttctcaccaaccctttgaactccgaaatgccgtgaacacatccccttagttggcattggtctactaaccgagtatcgtacaaagcgagacaggtctggcccctactgcttggatgcgaactaacagtgttcctatatccgtttatactgtggtcgatttcgaaccgatatctcaaagagagcgccggcagagccttagtatcggaactggcgagtgtgtttgggtgaggactattatgtggttggagtagagcgaatctcc

SEQ ID NO: 2 - DNA, 24 bp, Artificial Sequence
  Primer
gtagctccataatcagaagatgcg

SEQ ID NO: 3 - DNA, 27 bp, Artificial Sequence
  Primer
attcgctctactccaaccacataatag

SEQ ID NO: 4 - DNA, 26 bp, Artificial Sequence
  Detection probe
tccgatactaaggctctgccggcgct

SEQ ID NO: 5 - DNA, 24 bp, Artificial Sequence
  Primer binding site
gtagctccataatcagaagatgcg

SEQ ID NO: 6 - DNA, 27 bp, Artificial Sequence
  Primer binding site
ctattatgtggttggagtagagcgaat

SEQ ID NO: 7 - DNA, 26 bp, Artificial Sequence
  Detection probe binding site
agcgccggcagagccttagtatcgga

SEQ ID NO: 8 - DNA, 442 bp, Artificial Sequence
  Tag complementary sequence
acttcatcgaggtattagtcttctacgcgatggcatgtgttgccgccaaatttgaaacatctggagtgcgccgacgagtccagaacgcgtagggtatgaagtgtacctgcctttcgacacccaaacatagctattcggcatccaaggatcctagctcagtgggtttgttgaagctctcgagaagagtggttgggaaacttgaggctttacggcacttgtgtaggggaatcaaccgtaaccagatgattggctcatagcatgtttcgctctgtccagaccggggatgacgaacctacgcttgattgtcacaaggatataggcaaatatgacaccagctaaagcttggctatagagtttctctcgcggccgtctcggaatcatagccttgaccgctcacacaaacccactcctgataatacaccaacctcatctcgcttagagg

SEQ ID NO: 9 - DNA, 62 bp, Artificial Sequence
  Tag
tagttcacgggcaatgttcggccgttaattgttaacttaattgttaacacgggcaatacggg

SEQ ID NO: 10 - DNA, 62 bp, Artificial Sequence
  Tag
atcaagtgcccgttacaagccggcaattaagaattgaattaacaattgtgcccgttatgccc

SEQ ID NO: 11 - DNA, 21 bp, Artificial Sequence
  Primer
tagttcacgggcaatgttcgg

SEQ ID NO: 12 - DNA, 20 bp, Artificial Sequence
  Primer
cccgtattgcccgtgttaac

SEQ ID NO: 13 - DNA, 21 bp, Artificial Sequence
  Primer binding site
atcaagtgcccgttacaagcc

SEQ ID NO: 14 - DNA, 20 bp, Artificial Sequence
  Primer binding site
gttaacacgggcaatacggg

(19) United States
(12) Patent Application Publication
(10) Pub. No.: US 2024/0287620 A1
(43) Pub. Date: Aug. 29, 2024

(54) METHOD OF TAGGING FISH AND OTHER ANIMALS

(71) Applicant: SALMOTRACE AS, Tromsø (NO)

(72) Inventor: Dag HAUGSE, Oslo (NO)

(73) Assignee: SALMOTRACE AS, Tromsø (NO)

(21) Appl. No.: 18/569,829

(22) PCT Filed: Jun. 16, 2022
(86) PCT No.: PCT/EP2022/066495

§ 371 Date: Dec. 13, 2023

(30) Foreign Application Priority Data
Jun. 17, 2021 (GB) 2108684.8

(51) Int. Cl. C12Q 1/6888; A61D 7/00; A61K 39/00; A61K 39/385

(52) U.S. Cl. CPC C12Q 1/6888; A61D 7/00; A61K 39/385; A61K 2039/53

(57) ABSTRACT

The present invention relates to a method of tagging a non-human animal, particularly fish, said method comprising administering to said animal a tag nucleic acid molecule, wherein the tag nucleic acid molecule:
(i) comprises an ID sequence which is unique to the tag, which is non-coding and/or cannot be transcribed, and which may be distinguished from the ID sequences of other tag molecules, and
(ii) is detectable in or on said animal, or in a body tissue or fluid sample from said animal.

The tag may be used to identify the animal, for example in the context of tracking and tracing the animal. The tag may be administered to the animal in conjunction with a vaccine component, which may be provided as part of the tag nucleic acid molecule, or separately. Also provided herein are methods of tagging and vaccinating non-human animals, and combination products comprising a vaccine composition and a tag nucleic acid molecule, as well as apparatus for administering the tag nucleic acid molecule together with a vaccine.

### FIELD

The present disclosure, and the present invention, relates to methods of tagging a non-human animal, particularly fish, and in particular to methods of tagging comprising administering to said animal, or providing said animal with, a tag nucleic acid molecule. Methods of identifying a non-human animal and methods of vaccinating and tagging a non-human animal using such a tag molecule are also provided. Also provided and disclosed herein are combination products comprising a vaccine composition and a tag nucleic acid molecule, and such combination products for use in vaccinating and tagging a non-human animal. Such methods and products find utility in tracing and tracking of animals, particularly in the aquaculture industry, where they may be used to trace and track fish, particularly farmed fish.

### BACKGROUND

Traceability of non-human animals is desirable in many fields. In particular, in agricultural and food industries, such as fish farming, for example, it is useful to have systems to allow animals to be traced. This can allow the provenance of animals to be tracked and monitored, and guaranteed, and food safety issues to be managed and addressed, and can also provide additional confidence to consumers.

There are a number of existing traceability systems which may be used to identify and trace animals. Many of these may be classified as “paper-based” systems, whereby information regarding the provenance of a given animal or group of animals is tracked “on paper” or electronically. These systems include, for example, country of origin labelling or more specific food tracing information which may be provided on consumer packaging.

An alternative to paper-based systems is the use of “DNA-based” systems. Such systems typically involve collecting a sample from an animal either before or when the animal is slaughtered, from which the DNA of the animal can be isolated.

This DNA sample is then stored, together with all of the necessary information about the animal. When a trace is required, a sample is taken from the product in question, and the DNA is compared to the stored library of DNA samples to find a match. This can then be used to identify then retrieve the information stored about the animal with the original sample.

Animals are also commonly tracked using electronic tags, such as RFID chips. This can be done using ear tags, for example, or via smaller chips which can be injected into the body of the animal.

The present disclosure, and present invention, provides alternative tagging methods which allow for accurate identification and tracing of non-human animals, particularly fish, and more particularly farmed fish.

### SUMMARY

In a first aspect, provided and disclosed herein is a method of tagging a non-human animal, such as a fish, particularly a farmed fish, said method comprising administering to said animal a tag nucleic acid molecule, wherein the tag nucleic acid molecule:
(i) comprises an ID sequence which is unique to the tag, which is non-coding and/or cannot be transcribed, and which may be distinguished from the ID sequences of other tag molecules; and
(ii) is detectable in or on said animal, or in a body tissue or fluid sample from said animal.

In an embodiment the tag nucleic acid molecule is non-coding and/or cannot be transcribed. In another embodiment the tag nucleic acid molecule comprises a nucleotide sequence which comprises the ID sequence, which is non-coding and cannot be transcribed. In still another embodiment, the tag nucleic acid molecule may comprise a nucleotide sequence which is coding and/or which can be transcribed, in addition to the ID sequence, as long as the ID sequence itself is non-coding and cannot be transcribed. Accordingly, it is not an absolute requirement that the tag nucleic acid molecule in its entirety is non-coding and/or incapable of being transcribed.

Administration of the tag includes that the tag is, for example, injected or introduced to a site in or on the body of the animal in any way. This includes that the tag is provided in or on a carrier, which may be a solid carrier, which is administered or provided to the animal, for example inserted into a body site of the animal, or attached to an exterior body surface of the animal.

The tag nucleic acid molecules are distinguishable from each other and are detected in or on the animal, for example in a sample taken from the animal to which they were administered. Accordingly, they allow the animals to which they are administered to be identified and traced.

It will thus be understood that, following administration of the tag nucleic acid molecule to a given animal, the tag nucleic acid molecule may be detected in or on the body of the animal, for example in a body tissue or fluid sample from that animal. In this regard, the detection of the identity of the tag nucleic acid molecule may be used to identify the animal from which the sample was taken.

Accordingly, in a further aspect, provided herein is a method of identifying a non-human animal, such as a fish, particularly a farmed fish, said method comprising detecting in or on the animal, or in a body tissue or fluid sample from said animal, the presence and identity of a tag nucleic acid molecule, wherein the tag nucleic acid molecule has previously been administered to said animal, and comprises an ID sequence which is unique to the tag, which is non-coding and/or cannot be transcribed, and which may be distinguished from the ID sequences of other tag molecules.

It may be convenient in some embodiments to administer the tag nucleic acid molecule to the non-human animal whilst it is being vaccinated, i.e. to administer the tag nucleic acid molecule together with a vaccine composition or to co-administer the tag nucleic acid molecule and a vaccine composition. In a further embodiment, the vaccine component may be provided as part of the tag nucleic acid molecule. In other words, the tag nucleic acid molecule may comprise, in either order, a first portion, or a first nucleotide sequence, which comprises, consists of or is the ID sequence, and a second portion, or a second nucleotide sequence, which comprises, consists of or is a coding sequence encoding a vaccine component, or more particularly a vaccine antigen.

Thus, in a further aspect, also provided is a method of vaccinating and tagging a non-human animal, such as a fish, particularly a farmed fish, said method comprising administering to said animal (a) a vaccine composition together with a tag nucleic acid molecule or (b) a tag nucleic acid molecule which additionally comprises a nucleotide sequence encoding a vaccine component, wherein the tag nucleic acid molecule:
(i) comprises an ID sequence which is unique to the tag, which is non-coding and/or cannot be transcribed, and which may be distinguished from the ID sequences of other tag molecules; and
(ii) is detectable in or on said animal, or in a body tissue or fluid sample from said animal;
and wherein in (a) the vaccine composition and tag nucleic acid molecule are administered separately or in admixture.

In a further aspect, there is provided a combination product comprising:
(a) a vaccine composition for vaccination of a non-human animal; and
(b) a tag nucleic acid molecule which:
(i) comprises an ID sequence which is unique to the tag, which is non-coding and/or cannot be transcribed, and which may be distinguished from the ID sequences of other tag molecules; and
(ii) is detectable in or on said animal, or in a body tissue or fluid sample from an animal to which it has been administered.

This combination product may be referred to as a “vaccine-tag product”. Analogously, a tag nucleic acid molecule which includes, or incorporates, a nucleotide sequence encoding a vaccine component may be referred to as a “vaccine-tag nucleic acid molecule”.

In yet another aspect, the combination product as defined above may be for use in vaccinating a non-human animal, such as a fish, particularly a farmed fish, wherein said vaccine composition and tag nucleic acid molecule are provided for separate, sequential or simultaneous administration to said animal.

In another aspect, there is also provided a vaccine-tag nucleic acid molecule for use in vaccinating and tagging a non-human animal, such as a fish, particularly a farmed fish, wherein the vaccine-tag nucleic acid molecule comprises:
(a) a first nucleotide sequence which comprises an ID sequence which is unique to the tag, which is non-coding and/or cannot be transcribed, and which may be distinguished from the ID sequences of other tag molecules; and
(b) a second nucleotide sequence which encodes a vaccine component for vaccination of a non-human animal; and wherein the vaccine-tag nucleic acid molecule is detectable in or on said animal, or in a body tissue or fluid sample from an animal to which it has been administered.

Similarly, a yet further aspect provides the use of:
(a) a vaccine composition; and
(b) a tag nucleic acid molecule;
for the preparation of a combined product for use in vaccinating a non-human animal, such as a fish, particularly a farmed fish, wherein the tag nucleic acid molecule:
(i) comprises an ID sequence which is unique to the tag, which is non-coding and/or cannot be transcribed, and may be distinguished from the ID sequences of other tag molecules; and
(ii) is detectable in or on said animal, or in a body tissue or fluid sample from an animal to which it has been administered.

Another aspect provides use of a vaccine-tag nucleic acid molecule for the preparation of a vaccine-tag composition for use in vaccinating and tagging a non-human animal, such as a fish, particularly a farmed fish, wherein the vaccine-tag nucleic acid molecule comprises:
(a) a first nucleotide sequence which comprises an ID sequence which is unique to the tag, which is non-coding and/or cannot be transcribed, and which may be distinguished from the ID sequences of other tag molecules; and
(b) a second nucleotide sequence which encodes a vaccine component for vaccination of a non-human animal;
and wherein the vaccine-tag nucleic acid molecule is detectable in or on said animal, or in a body tissue or fluid sample from an animal to which it has been administered.

In certain embodiments, it may be desirable to use a specific injection apparatus to administer the tag nucleic acid molecule with the vaccine composition to the non-human animal, which may in some embodiments be a fish, particularly a farmed fish.

Thus, a still further aspect provides a vaccine injection apparatus which has been adapted for administering a tag nucleic acid molecule with a vaccine composition (which may be provided together in the form of a combination product referred to as a vaccine-tag product) to a non-human animal. The injection apparatus may comprise a first feed of a vaccine composition and a second feed of a tag nucleic acid molecule, wherein the injection apparatus is configured to administer the tag nucleic acid molecule when administering the vaccine composition to the non-human animal.

In one embodiment, a primary input port may be provided on an injector of an injection apparatus to connect with the first feed of vaccine composition, and a secondary input port may be provided on the injector to connect with the second feed of tag nucleic acid molecule. The injector may comprise a mixing chamber to mix the tag nucleic acid molecule with the vaccine composition prior to administering the vaccine composition, or the first and second feeds may be kept separated.

The administering of the tag nucleic acid molecule may take place concurrently with the vaccine composition, in the sense of during the course of a single process, such as a single injection cycle of the vaccine composition. For example, the injection apparatus may be configured to administer the tag nucleic acid molecule and the vaccine composition concurrently, such that administering the tag nucleic acid molecule may take place within 20 minutes of administering the vaccine composition (which may be administered before or after the tag nucleic acid molecule), preferably within a minute of administering the vaccine composition, and more preferably in a much shorter time frame such as simultaneously with the vaccine product, i.e. administering the tag nucleic acid molecule and the vaccine composition at the same time as or at substantially the same time as each other, e.g. within 1 minute of each other. The terms “concurrent administration” and “simultaneous administration” are defined further below. In some embodiments the tag nucleic acid molecule and the vaccine composition may be administered in admixture as a single formulation.

In preferred embodiments the tag nucleic acid molecule may be administered at the same time as the vaccine composition, i.e., simultaneously with, or at least during an injection cycle while the non-human animal is being restrained for administering the vaccine composition. In this way the non-human animal is restrained for a minimum amount of time and the administering of the tag nucleic acid molecule does not unduly slow the administering of the vaccine composition.

However, it is also envisaged that a tag nucleic acid molecule may be administered before and/or after the administering of the vaccine composition, as the non-human animal passes through or past the injection apparatus. For example, a non-human animal, such as a fish, may pass along a conveyance system, and in so doing, may pass through a first injection station where one of a tag nucleic acid molecule or a vaccine composition is administered, and then pass through a second injection station where the other of the tag nucleic acid molecule or vaccine composition is administered.

In more preferred embodiments, the injection apparatus may be configured to administer the tag nucleic acid molecule and the vaccine composition to a same point of injection on the non-human animal. In this way it may be possible to reduce potential for damage to the non-human animal. In some embodiments, the non-human animal may be a fish, particularly a farmed fish. The tag nucleic acid molecule may be administered through the same needle as the vaccine composition. For example, the injection apparatus may be configured to administer the tag nucleic acid molecule in admixture with the vaccine composition in a mixed solution, e.g., at the point of delivery to the non-human animal, or configured to administer the tag nucleic acid molecule simultaneously with the vaccine composition via a second lumen of the needle allowing the tag nucleic acid molecule and the vaccine composition to be kept separated, or configured to administer the tag nucleic acid molecule through the same needle as the vaccine composition at a sequential point in time, for example, before or after a dose of vaccine composition has been administered to the non-human animal.

Administering the tag nucleic acid and vaccine composition to a same point of injection on the non-human animal may also include injection to a localised area of the non-human animal through two needles which are in close proximity, for example, within a separation of less than 5 mm and preferably less than 3 mm.

In some instances, for example, where it might benefit the configuration of the injection apparatus, administration of the tag nucleic acid molecule and the vaccine composition may occur at different points of injection on the non-human animal, e.g., through separate injectors of the injection apparatus.

Depending on imposed conditions of any regulatory approval of the vaccine composition, it may be an option to mix the tag nucleic acid molecule with the vaccine composition prior to supplying the vaccine composition to the injection apparatus. The vaccine composition may be packaged in a way to facilitate such mixing of the tag nucleic acid molecule with the vaccine composition, e.g., on site in a location where the non-human animal is due to be vaccinated shortly before the vaccine composition is administered.

Thus another aspect provides a pharmaceutical package, which may be in the form of a bag or other sealed container, comprising a first compartment containing a vaccine composition for a non-human animal and a second compartment containing a tag nucleic acid molecule, wherein the pharmaceutical package is arranged to allow mixing of the tag nucleic acid molecule with the vaccine composition, e.g., on site in a location where the non-human animal is due to be vaccinated, prior to administering the vaccine composition to the non-human animal. This mixing may be achieved through rupture of one of the compartments to allow the tag nucleic acid molecule to infiltrate the vaccine composition. The compartment may comprise a frangible or tearable wall separating the tag nucleic acid molecule from the vaccine composition. In another embodiment, this mixing may be achieved through opening of a closure sealing off one or both of the first and/or second compartments.

The tag nucleic acid molecule may: (i) comprise an ID sequence which is unique to the tag and may be distinguished from the ID sequences of other tag molecules, and (ii) be detectable in or on said animal, or in a body tissue or fluid sample from an animal to which it has been administered, wherein said ID sequence is non-coding and/or cannot be transcribed. The tag nucleic acid molecule may be present in a tag nucleic acid molecule product, for example, in a solution of such tag nucleic acid molecules, e.g., as a nucleic acid tracer product, which can be combined easily with the vaccine composition (e.g., through being soluble in the vaccine composition) to form an admixture that can be administered to the non-human animal.

From yet a further aspect, there is provided a system for administering a vaccine-tag product to a non-human animal, such as a fish, particularly a farmed fish, the system comprising an injection apparatus and a supply of vaccine-tag product for administering to the non-human animal, wherein the vaccine-tag product is a combination product comprising:
(a) a vaccine composition for vaccination of a non-human animal; and
(b) a tag nucleic acid molecule which
   (i) comprises an ID sequence which is unique to the tag and may be distinguished from the ID sequences of other tag molecules, and
   (ii) is detectable in or on said animal, or in a body tissue or fluid sample from said animal to which it has been administered,
   wherein said ID sequence is non-coding and/or cannot be transcribed.

### FIGURES

Certain preferred embodiments will now be described in greater detail and by way of example only with reference to the accompanying drawings, in which:

FIG. 1 shows a schematic representation of an injection apparatus;

FIG. 2 shows a schematic representation of a pharmaceutical package.

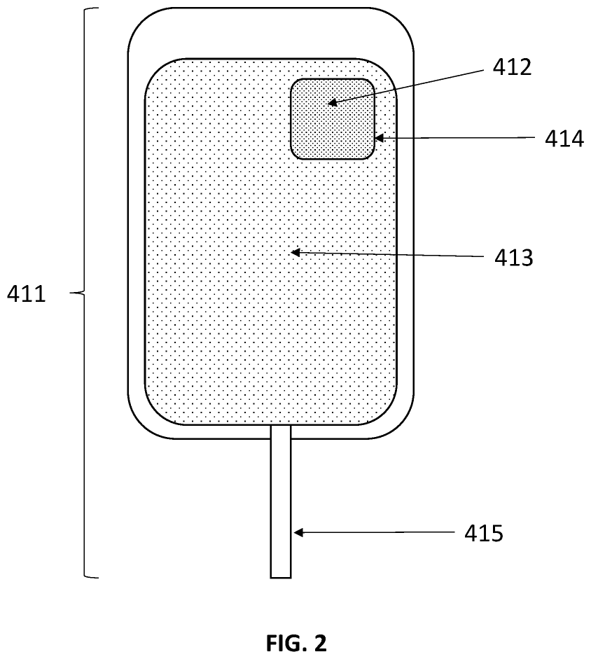

### DETAILED DESCRIPTION

The present invention involves the use of a nucleic acid molecule as a tag to identify or trace a non-human animal, such as a fish, particularly a farmed fish. The term “tagging” broadly means labelling or marking an animal such that it can be identified and tracked or traced. The tag allows the animal to be monitored. This may be as a live animal, and/or following slaughter of the animal as a “meat” or food product. The term “meat” is used broadly herein to include the edible flesh or tissue of any edible animal, including particularly fish or other aquatic animals. The tag may alternatively be termed a “tracer”. The tag nucleic acid molecule is thus used as means of “encoding” (not in the conventional sense of the nucleic acid code), or providing, imparting or reporting information about the animal. The use of nucleic acid allows a vast, almost unlimited, multiplicity of different tags (more specifically tag sequences) to be generated, and thus for a very large degree of information to be provided, whether in relation to the number of animals tagged, or in relation to the information provided about an animal.

The tag may identify the animal. As will be discussed in more detail below, an animal may be identified at different levels. This includes that the animal is identified as an individual animal, but is not limited to this. In other embodiments, the animal may be identified at the level of, i.e. as belonging to, a group, batch, set, or population of animals. In other words, the identity which is determined may not be that of an individual animal, but of a population, or other group or cohort, of animals, as indicated above. For example, the source of the animal, e.g. a farm, may be identified, or even a particular site or time in the farm that the animal was raised, reared or sold etc.

The tag nucleic acid molecule may be comprised of any suitable nucleic acid. For example, the tag nucleic acid molecule may be comprised of DNA or RNA, or a combination thereof. In some embodiments, the tag nucleic acid molecule may alternatively, or additionally, comprise one or more artificial or synthetic nucleotides (i.e. nucleotide analogues), such as Peptide Nucleic Acid (PNA), Locked Nucleic Acid (LNA), GNA, XNA and/or TNA. In a particular embodiment, the tag nucleic acid molecule is DNA. The DNA may be modified, for example to add protecting or blocking groups, as discussed further below.

It will be understood that the term “a tag nucleic acid molecule” as used herein does not imply the use of a single molecule, and in line with common usage refers to a species of molecule. In practice, a multiplicity of tag nucleic acid molecules will be used to tag an individual animal, in the sense of multiple copies of the tag molecule. The tag nucleic acid molecule may be provided as a population of multiple copies of the molecule, conveniently as a population of amplicons of a tag nucleic acid molecule sequence. Generally speaking, the individual molecules in such a population will be identical, or will be expected to be identical. However, as with all biological processes, absolute precision of copying cannot be guaranteed, and there may be some minor, or insignificant or negligible changes. Any such changes to the sequence of molecules within a population would be de minimis. As noted above, the tag nucleic acid molecule may alternatively be referred to as a nucleic acid tracer.

It is generally suitable to administer a large number of copies to the animal, to help ensure persistence of the tag molecule on or within the animal. This may readily be accomplished when the tag nucleic acid molecule is prepared as an amplicon. Typically, a preparation of amplicons of a tag nucleic acid molecule prepared by a typical amplification process may comprise millions, or up to a billion, or more copies of the molecule. In some embodiments, all copies of the molecule may be identical. Tag nucleic acid molecules for use as described herein may conveniently be prepared by PCR, but any desired or convenient amplification method may be used, including LCR, and RCA, or any of the other amplification methods described below.

The tag nucleic acid molecule may be a single or double stranded nucleic acid molecule. In a particular embodiment, the tag nucleic acid molecule is double stranded. The tag nucleic acid molecule may be linear or circular.

The tag nucleic acid molecule as defined herein comprises an ID sequence which is unique to the tag and may be distinguished from the ID sequences of other tag molecules. It is not necessary for any given ID sequence to be unique in itself, that is previously unknown or unreported, as long as it can be distinguished from the ID sequences of other tags. However, this is not excluded, and in an embodiment the ID sequences of some or all of the tags may be unique absolutely. The ID sequence is detected in order to detect and identify the tag, and therefore the animal. Thus, the ID sequence is of a length which allows it to be detected. The detection of nucleic acids is known in the art, and a variety of procedures are available to detect a nucleic acid, including sequencing, size separation, or the binding of detection probes, the binding of which is detected to detect the nucleic acid, or through amplification or extension reactions using one or more primers which are specific for, or which flank and allow the amplification of the target sequence in question (here the ID sequence). As will be discussed in more detail below, any convenient or desired means or method of nucleic acid detection may be used to detect the tag molecule. However, conveniently, the ID sequence may be capable of binding a detection probe, in order to facilitate the detection and identification of the tag nucleic acid molecule. In some embodiments, the ID sequence may be 6-30 nucleotides in length, such as 6-28, 6-26, 8-30, 8-28, 8-26, 8-25, 8-20, 10-18, or 12-16 nucleotides in length. It may however be longer than this, depending on the number of tags, and the method of detection etc. In some embodiments, the ID sequence may be 10-12 nucleotides in length. It will be understood that there are 1,049,576 (4¹⁰) different possible tags having an ID sequence comprising 10 nucleotides, and over 4.2 million (4¹¹) possible tags having an ID sequence comprising 11 nucleotides. It is anticipated that this number of potential different tags will be sufficient for the presently disclosed methods.

As will be discussed in more detail below, the tag nucleic acid molecule will generally be of a limited length, for example less than 100 nucleotides. It may be longer where the tag molecule additionally comprises a coding sequence. The tag nucleic acid molecule may thus alternatively be referred to as a tag oligonucleotide.

The ID sequence of the tag nucleic acid molecule disclosed herein is non-coding. By “non-coding”, it is meant that the ID sequence of the tag nucleic acid molecule does not encode a protein sequence. In this regard, the tag nucleic acid molecule may comprise a synthetic, artificial sequence, or a non-natural or non-native sequence.

Additionally, or alternatively, the ID sequence cannot be transcribed, i.e. the ID sequence cannot give rise to transcription or cannot serve to allow a RNA transcript of the molecule to be generated. In an embodiment, the ID sequence of the tag nucleic acid molecule comprises one or more stop codons at or near the 5′ end, to prevent transcription from occurring. For example, there may be 1, 2 or 3 stop codons present at the 5′ end. The stop codon may be selected from TGA, TAA, TAG, or any other suitable stop codon. In some examples, the stop codon may be TGA. If multiple stop codons are used, the stop codons may be contiguous.

For detection, amplification of the tag nucleic acid molecule, or of the ID sequence thereof may be convenient or desirable. Accordingly, the tag nucleic acid molecule may comprise at least one primer binding site. Although in some embodiments, the ID sequence may be detected through the binding of a specific primer, that is a primer which binds specifically to the ID sequence, and the detection of an amplification product, conveniently amplification is used as a preliminary step, to amplify the ID sequence, which is then subsequently detected in a separate or further step. Thus, the ID sequence may be amplified using one or more primers binding at one or more primer binding sites in the tag molecule, and the ID sequence, or complement thereof, in the amplicon may then be detected, for example by sequencing or binding of a detection probe. The tag molecule may thus comprise one or more primer binding sites and an ID sequence. In some embodiments, the tag nucleic acid molecule comprises two primer binding sites. In some embodiments, the ID sequence may partially or fully overlap with at least one of the primer binding sites. Alternatively put, in some embodiments, the ID sequence may partially or fully comprise at least one primer binding site. In another embodiment the primer binding site(s) are separate to the ID sequence (that is they are physically separated, i.e. they do not overlap).

In one particular embodiment, the tag nucleic acid molecule comprises two primer binding sites which flank the ID sequence, such that the tag nucleic acid molecule may be considered to have a structure comprising (from 5′ to 3′): a first primer binding site; an ID sequence; and a second primer binding site. Alternatively put, the tag nucleic acid molecule may comprise an ID sequence between the primer binding sites.

The primer binding sites within the tag nucleic acid molecule are capable of binding of a primer to allow the tag nucleic acid molecule (particularly the ID sequence of the tag nucleic acid molecule) to be amplified. In some embodiments, the tag nucleic acid molecule comprises a first primer binding site for binding a forward primer and a second primer binding site for binding of a reverse primer.

In some embodiments, the primer binding sites may be 16-30 nucleotides in length. The primers may similarly be 16-30 nucleotides in length. It will be understood that the primers will typically be of the same length as the primer binding sites. This length of primer is sufficient to ensure that the primers will only hybridise to the intended primer binding sites in the tag nucleic acid molecule, and will not hybridise to other sequences, such as DNA sequences in the cells of the non-human animal to which the tag nucleic acid molecule is administered.

The primer binding sites may be separated from each other by a sequence of at least 6 nucleotides, so that the two primer binding sites do not overlap. In a preferred embodiment, the primer binding sites are separated from each other by a sequence of at least 6, 7, 8, 9, or 10 nucleotides, such as at least 15, at least 20 or at least 25 nucleotides. In some embodiments, the primer binding sites may be separated from each other by a sequence having no more than 60 nucleotides. Alternatively, the primer binding sites may be adjacent to each other, i.e. there may be no intermediate nucleotides between the primer binding sites. This arrangement is particularly suitable where the tag nucleic acid molecule is to be detected by a method which relies on ligation, such as ligation chain reaction (as discussed below), as the primers are positioned such that they can be ligated together.

Conveniently, the primer binding sites may be common primer binding sites. That is, they may be common between all, or a sub-set of, tag nucleic acid molecules. This is convenient as it allows the same primers to be used to amplify the ID sequences of all or a number of tag molecules.

In total, the tag nucleic acid molecule may be at least 36 nucleotides in length, such as at least 40, 45, 50, 55, 60, 65 or 70 nucleotides. In an embodiment, the tag nucleic acid molecule may comprise no more than 1000 nucleotides, such as no more than 900, 800, 700, 600, 500, 400, 300, 200, 100, 95, 90, 85 or 80 nucleotides. Thus, the tag nucleic acid molecule may comprise, for example, 36-500 nucleotides, such as 40-400, 50-300, 55-200, or 60-100 nucleotides.

The tag nucleic acid molecule may be linear or circular. In either case, in certain embodiments the tag nucleic acid molecule may not have any secondary structure, including loops, stem loops, hairpins, pseudoknots or any other secondary structures. In certain embodiments, the tag nucleic acid molecule may have a coiled or supercoiled structure. In an embodiment, the tag nucleic acid molecule may be a single stranded linear nucleic acid (e.g. DNA). Alternatively, the tag nucleic acid molecule may be provided in partially or fully double-stranded form. In an embodiment, it may be provided as a mixture of two linear complementary DNA strands. In an embodiment the tag nucleic acid molecule may be a circular molecule. A circular molecule may have a coiled or supercoiled structure.

In an embodiment, the circular molecule may be a plasmid. Thus, the tag nucleic acid molecule may be comprised within a plasmid. In an embodiment the plasmid does not comprise a nucleotide sequence capable of being expressed, that is, it does not comprise a coding sequence, or a sequence capable of being transcribed. However, in another embodiment, the plasmid may comprise a coding sequence capable of being transcribed, e.g. a sequence encoding a vaccine component. In another embodiment, the circular molecule which comprises the tag nucleic molecule is akin to a plasmid (i.e. is “plasmid-like”) but is not capable of independent replication, e.g. it does not comprise an origin of replication. This may particularly be the case where the circular molecule does not contain a coding sequence, or a sequence capable of being transcribed. The plasmid or plasmid-like circular molecule may be coiled, or more particularly, supercoiled.

To enable a circular molecule, e.g. plasmid or plasmid-like molecule, to be formed, the molecule will need to be of length which permits this. For a nucleic acid circle, this may be of a size of at least 80 nucleotides or base pairs (bp), e.g. at least 100, 110, 120, 130, 140, 150, 160, 170, 180, 190 or 200 nucleotides or bp. For a plasmid or plasmid-like molecule, a longer size may be needed, e.g. at least 600, 650, 700, 750, 800, 820, 830, 840, 845, or 850 bp. Thus, a circular molecule, particularly a plasmid or plasmid-like molecule, may comprise additional nucleotide sequence(s) to allow the molecule to be formed. Alternatively viewed, the tag nucleic acid molecule may be provided with, or may comprise, one or more additional nucleic acid sequences to increase size, in order to allow the molecule to form (i.e. be generated or produced).

In some embodiments, it may be desirable to select or design the tag molecule so as to achieve a given or desired melting temperature or to alter the melting temperature of the tag nucleic acid molecule, in order to control the stability of the tag nucleic acid molecule. In some embodiments, the melting temperature of the tag nucleic acid molecule may be at least 60° C., such as at least 65, 67, 70, 72, 75, 77, 80, 82 or 85° C. In some embodiments, the tag nucleic acid molecule may have a melting temperature between 70 and 90° C. The melting temperature of the tag nucleic acid molecule may be selected or adjusted based on the method by which it is intended to detect the tag nucleic acid molecule. Such detection methods are described in more detail below, and the skilled person would understand how to select a suitable melting temperature for a given detection method.

Higher melting temperatures may be achieved by providing a higher GC content in the sequence of the tag nucleic acid molecule. In some embodiments, the tag nucleic acid molecule may have a GC content of at least 50%, such as at least 55, 60, 65, 70 or 75%. In some embodiments, the tag nucleic acid molecule may have a GC content of 50-60%.

As noted above, the ID sequence of the tag nucleic acid molecule is non-coding and/or cannot be transcribed. In addition to the ID sequence, the tag nucleic acid molecule may contain a sequence which is coding and/or which can be transcribed. Accordingly, the tag nucleic acid molecule may comprise a coding region, comprising the sequence which can be transcribed, and a non-coding region, comprising the ID sequence. This coding region of the tag nucleic acid molecule may comprise any sequence which it is desired to express in the host animal. For example, the tag nucleic acid molecule may comprise a sequence which is to be expressed as part of a vaccine, e.g. it may encode a vaccine component, e.g. a vaccine antigen.

This coding region of the tag nucleic acid molecule, if present, may be upstream or downstream of the non-coding region comprising the ID sequence. In order to ensure that the ID sequence is not transcribed, at least one stop codon will be present 5′ of the ID sequence. For example, if the coding region is upstream of the non-coding region, it will be followed by at least one stop codon before the beginning of the ID sequence, to ensure that the ID sequence is not transcribed.

Alternatively, in an embodiment, the tag nucleic acid molecule in its entirety is non-coding, i.e. it does not encode a protein sequence. In this regard, the tag nucleic acid molecule may comprise a synthetic, artificial sequence, or a non-natural or non-native sequence. In some embodiments, the tag nucleic acid molecule may comprise a sequence which does not occur in nature, or in the non-human animal to be tagged.

In some embodiments it may be advantageous to select the tag nucleic acid molecule such that the sequence of the tag nucleic acid molecule, or a significant portion thereof, particularly the ID sequence, is not present in the genome of the animal to which the tag is to be administered. This ensures that the DNA of the host animal is not amplified when the tag is detected. This selection of an appropriate tag sequence (or ID sequence) may be done by comparing the intended sequence with known genome sequences for the relevant host animal.

In some embodiments the tag nucleic acid molecule cannot be transcribed. That is, it is not capable of undergoing transcription, or cannot serve to allow a RNA transcript of the molecule to be generated. In an embodiment, as noted above, the tag nucleic acid molecule comprises one or more stop codons at or near the 5′ end, to prevent transcription from occurring. For example, there may be 1, 2 or 3 stop codons present at the 5′ end. For example the stop codon may be TGA. In an embodiment, the stop codons may be contiguous.

Advantageously, the tag nucleic acid molecule can be designed to ensure that it is stable, such that it is detectable, for example that it can be detected in a body tissue or fluid sample from the animal to which it is administered. Moreover, it will be understood that the tag nucleic acid molecule may be capable of being detected several times at different time periods, i.e. from several different samples taken from the animal at different times, such that the animal can be identified and traced at any point in time after the administration of the tag. In a preferred embodiment, the tag nucleic acid molecule is capable of being detected at any point after administration. Stability of the molecule can be increased in various ways, including by consideration of its sequence (e.g. GC content etc.) and of its structure, and also by including modifications which render it resistant to degradation as discussed further below.

Persistence of the molecule may also be enhanced by administering it in high copy number. As noted above, in a preferred embodiment, the tag nucleic acid molecule is provided to the animal as a population of amplicons, i.e. multiple amplification products. Accordingly, as a significant number of copies of the tag nucleic acid molecule are provided to the animal, it may be possible for a proportion of the tag nucleic acid molecules which are provided to be degraded, without compromising the detectability of the tag nucleic acid molecule. Nevertheless, in order to remain detectable for extended periods of time, it is desirable that the tag nucleic acid molecule is resistant to degradation. Accordingly, the tag nucleic acid molecule may comprise one or more additional nucleotides at each end to protect from exonuclease degradation. In some embodiments, the tag nucleic acid molecule may comprise one or more modified nucleotides which are exonuclease-resistant. For example, the tag nucleic acid molecule may comprise an exonuclease block comprising a modified nucleotide which is resistant to exonuclease degradation on both ends of each strand that is present in the molecule. The tag nucleic acid molecule, or at least the ID sequence thereof, is advantageously also resistant to degradation by endonucleases. Methods and modified nucleotides for use in conferring nuclease resistance are known and available in the art. Various exonuclease blocks, comprising modified linkages, blocking groups or modified nuclease resistant nucleotides are widely known and used in the art, and any of these could be used. Accordingly a nucleotide or nucleoside may be modified to include or incorporate a blocking group which may inhibit an enzyme (e.g. an exonuclease) from functioning, e.g. from binding. For example an exonuclease block may comprise one or more nuclease-resistant nucleotide residues. These may for example be 2′O-modified-RNA residues, LNA residues, PNA residues, phosphothiate-modified nucleic acids, or a polyethylene-linker backbone moiety incorporated in between nucleotide residues. Abasic (apurinic or apyramidinic, or AP) sites may also be included as exonuclease blocks

By way of representative example, as a convenient way to confer resistance to exonuclease digestion the nucleic acid molecule may comprise phosphorothioate bonds, or 2′-modified nucleosides, for example 2′-O-methyl or 2′-O-methoxyethyl nucleosides, which may be included for example at the termini of the tag nucleic acid molecule. Other modifications to confer nuclease resistance include inverted deoxythymidine bases and dideoxnucleotides. Other modifications may be attached through linkers at the 5′ and/or 3′ ends, such as biotin or other hapten groups or blocking groups, but these are less preferred.

In some embodiments, the sequence of the tag nucleic acid molecule may be arranged so as to avoid including recognition sites for restriction enzymes or endonucleases. Restriction endonucleases, and their recognition and/or cleavage sites, are well known and widely reported in the literature, and thus it is a straightforward matter for a skilled person to screen nucleotide sequences, and or to design them, to avoid the presence of such sites.

The skilled person will appreciate that nucleic acid molecules which are administered to animals, particularly fish, may persist for an extended period of time, for example, for at least 1 year, such as at least 2, 3, 4 or 5 years or more. In this regard, it is noted that it has been reported that an expression plasmid which was administered to salmon was detectable in the fish after a time period of 535 days (Tonheim et al, 2007, Fish and Shellfish Immunology 23, 867-876). It will thus be understood that the tag nucleic acid molecule disclosed herein can be expected to be sufficiently stable and resistant to degradation to persist in the animals to which it is administered.

As noted above, one strategy to improve resistance to degradation is to present the tag nucleic acid molecule as a coiled or supercoiled structure. This may readily be achieved in the context of a circular nucleic acid molecule, akin to a plasmid. Thus, as a strategy to increase stability or persistence of the tag nucleic acid molecule, it may be provided in the form of a plasmid or a plasmid-like molecule, i.e. a circular nucleic acid molecule which is not capable of independent replication, e.g. does not comprise an origin of replication.

The tag nucleic acid molecule as defined above is to be administered to a non-human animal. The animal to which the tag nucleic acid molecule is administered may be a farmed animal. In some embodiments, the animal is a mammal. In some embodiments, the animal may be a livestock animal, such as a cow or bull, sheep, pig, horse, donkey, goat, camel, llama, alpaca, or rabbit, a poultry animal, e.g. a chicken, duck or turkey, or a domestic or sports animal, e.g. a cat, dog, or horse, or a laboratory animal (e.g. a rodent such as a mouse, rat or guinea pig). The animal may thus be a murine, feline, canine or equine species.

The tagging method herein may also find utility in the tagging of animals in the context of conservation, or the protection of endangered species, or wild animals in general. The animal may be thus be a wild animal, or a zoo animal, such as for example an elephant, tiger, lion, rhinoceros, polar bear, seal, sea-mammal, and this may include non-mammalian animals, for example a bird (e.g. migratory bird), etc.

More generally, in some embodiments, the animal may be a non-mammalian animal, for example a non-mammalian aquatic animal. In this regard, the animal may be a fish (or alternatively expressed, a piscine animal or species), a mollusc or a crustacean. More specifically, the animal may be a fish. It may be a fish of any species or genus. For example, the animal may be selected from a salmon, tuna, cod, sea trout, pangasius, tilapia or seabass. In some embodiments, the fish may be a cold water fish. In other embodiments, it may be a warm water fish. It may be a fresh- and/or salt-water fish. In some embodiments, the animal may be a fish which is suitable for keeping or which is intended to be kept in an aquarium (referred to herein as an “aquarium fish”). The animal may be a mollusc selected from an oyster, clam, scallop or mussel, or an octopus or squid. The animal may be a crustacean selected from a crab, lobster, shrimp, prawn or crawfish.

In a representative embodiment, the animal is a fish, particularly a farmed fish. In a more particular embodiment, the animal is a salmon.

It will be understood that the method by which the tag nucleic acid molecule is administered to the non-human animal will depend on the type of animal in question. The tag nucleic acid molecule may be administered in any convenient or desired manner, and the skilled person will be capable of selecting an appropriate method of administration for a given animal.

In some embodiments, the tag nucleic acid molecule may be administered by injection, infusion or implantation. The administration may be made to any desired, convenient or appropriate site in or on the animal body. This may be any tissue or cavity in or on the body. For example, the cavity may be the oral cavity, the peritoneal cavity or the abdominal cavity etc. In a representative embodiment, the administration may be made into the abdominal cavity, or into the musculature (muscle tissue) or skin of the animal, for example by intramuscular injection, or injection into the abdominal cavity. In some embodiments, the injection may be a subcutaneous injection, an intradermal injection or an intravenous injection. It may be desirable to inject the non-human animal with the tag nucleic acid molecule in such a manner that it does not cause damage to the animal, or any other visible effects which could reduce the value of the animal. In a particular embodiment, the animal is a fish, and the tag nucleic acid molecule is administered via an injection into the abdominal cavity or the muscle tissue.

The tag molecule may be provided or formulated for administration in any convenient or desired manner according to principles well known and practiced in the art. Thus, for example, a composition comprising the nucleic acid tag molecule may be prepared comprising the tag molecule and one or more carriers or excipients. For example, the tag nucleic acid molecule may be formulated, or provided, in a suitable liquid vehicle. Suitably, this may be an aqueous vehicle, i.e. an aqueous formulation or composition may be provided. This may be e.g. water or a buffer solution. Additives may be added, for example to aid or facilitate storage of the tag molecule. It will be understood that the carriers, excipients or additives etc. may be suitable for administration to the animal, and may thus be pharmaceutically or physiologically suitable for such administration.

In some embodiments, the tag nucleic acid molecule may be formulated or provided in nuclease-free water, or in a suitable buffer such as Tris-EDTA (also known as TE buffer), Tris-Acetate-EDTA (TAE buffer), Tris-Borate-EDTA (TBE buffer) or lithium borate (LB buffer). The tag nucleic acid molecule composition may also comprise one or more additives, for example to control the stability of the tag nucleic acid molecule or the viscosity of the composition. Suitable additives are well known in the art and include, for example, hydrophobic salts such as lidocaine, mepivacaine, and prilocaine.

In other embodiments, a solid phase carrier for the tag nucleic acid molecule may be used. Thus, the tag nucleic acid may be provided in or on a solid support, or in other words a solid carrier. Accordingly, the tag nucleic acid molecule may be incorporated into, or attached (e.g. conjugated, or otherwise linked or bound to) a solid carrier or support. Methods and protocols for immobilising nucleic acids and attaching them to a solid support are well known in the art, and include covalent and non-covalent bonding, cross-linking, and encapsulation etc. The solid support or solid carrier may be selected from any suitable such support or carrier known in the art. For example, the tag nucleic acid molecule be linked to superparamagnetic beads, which subsequently may be conveniently isolated from a sample using methods known in the art, or to a small plastic pin of, for example, approximately 1 cm in size or smaller. A carrier comprising or carrying the tag nucleic acid material may be administered to the animal. As noted above, administration in this respect includes any means or method of providing the carrier carrying the tag nucleic acid molecule to the animal. In some embodiments, the carrier may take the form of a strip or similar to which nucleic acid molecules are bound (or in other words, in or on which they are immobilised). Depending on the identity of the carrier and of the non-human animal, the carrier may be inserted, e.g. implanted into the animal, or it may be otherwise attached to the animal. Thus, the solid carrier may be a partially or fully in-dwelling implantable device, it may be attached to the animal as a piercing (e.g. an ear tag or a fin tag in a fish) or it may be worn by the animal (e.g. a cuff or ring around a limb) or inserted into the animal (e.g. implanted under the skin of an animal, such as a fish). It will be understood from the above that a solid carrier may be internal or external to the animal body.

The solid support may be composed of any suitable material, depending on how and where it is administered or provided to the animal. Conveniently, it may be composed of a plastic or polymer material.

The tag nucleic acid may be administered to the animal at any desired or convenient life stage, depending on the animal and the purpose of the tagging. Thus, the tag may be administered to infant, juvenile or adult animals. In the case, of fish, for example, it may be administered at the smolt stage.

It may be expedient and convenient to administer the tag at the time of, or concurrently with, vaccination. Many non-human animals are vaccinated in the course of animal husbandry. Accordingly, in a further aspect, as noted above, there is provided a method of vaccinating and tagging a non-human animal comprising administering the tag nucleic acid molecule to said animal together with a vaccine.

In this regard, also provided is a combination product comprising a vaccine composition for vaccination of a non-human animal, and a tag nucleic acid molecule as defined herein. This combination product may be referred to as a “vaccine-tag product”. In some embodiments, this combination product may be for use in vaccinating a non-human animal. Also provided is the use of a vaccine composition and a tag nucleic acid molecule as defined herein for the preparation of a combined product for use in vaccinating a non-human animal.

As noted above, livestock and other farmed animals and indeed domestic and sports animals are routinely vaccinated, and thus it may be convenient to administer the tag nucleic acid molecule during the vaccination process, i.e. together with a vaccine, or indeed that tag and vaccine component may be combined in a single nucleic acid molecule (a “vaccine-tag” nucleic acid molecule). This is particularly the case for farmed fish, where vaccinations are common, and where vaccines can be administered by automated machines which isolate, sort and vaccinate the fish. Alternatively, the administration may be performed manually. This may be achieved using devices such as injector devices which permit multiple delivery of injections to facilitate the administration. In the case that a single vaccine-tag molecule is used, conventional and available equipment may be sued for administration. However, where the tag and vaccine are separate components, the process of vaccination may be modified to additionally include the co-administration of a tag nucleic acid molecule. In the case of fish, the vaccine is frequently administered by injection, and accordingly, the tag nucleic acid molecule may be co-administered by injection. Where the tag nucleic acid molecule is to be administered in the form of a solid carrier comprising the tag nucleic acid molecule, this carrier may similarly be injected or otherwise attached or administered to the fish during the vaccination process.

In this regard, also provided herein is a vaccine injection apparatus which has been adapted for administering a tag nucleic acid molecule with a vaccine composition to a non-human animal.

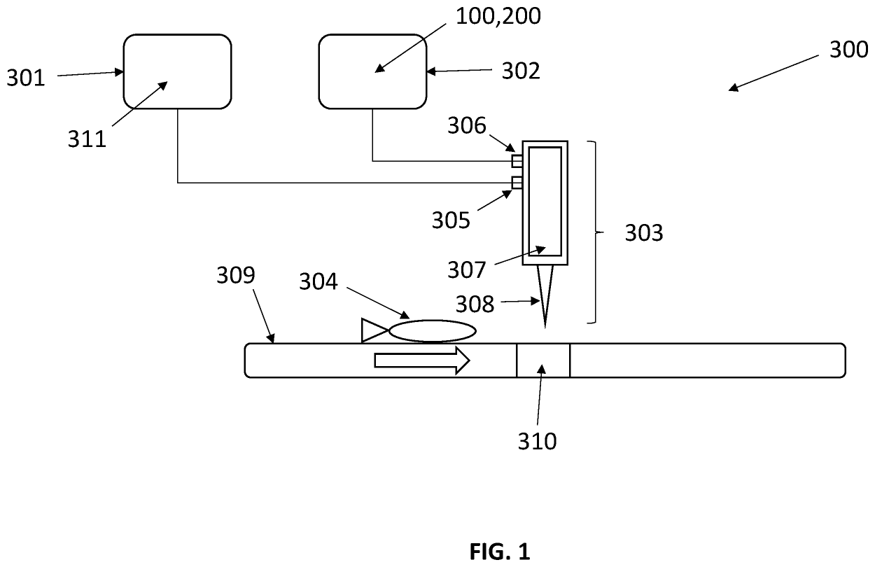

As shown in FIG. 1, the injection apparatus 300 may comprise a first feed 301 of a vaccine composition 311, a second feed 302 of a tag nucleic acid molecule 100,200, and an injector 303. The first feed and second feed 301,302 are operably connected to the injector 303. In addition, the injection apparatus 300 is configured to administer the tag nucleic acid molecule 100,200 when administering the vaccine composition to the non-human animal 304. In the embodiment, a primary input port 305 is provided on the injector 303 of the injection apparatus 300 to connect with the first feed 301 of vaccine composition 311, and a secondary input port 306 is provided on the injector 303 to connect with the second feed 302 of tag nucleic acid molecule 100,200. The injector 303 may comprise a mixing chamber 307 as shown to mix the tag nucleic acid molecule 100,200 with the vaccine composition 311 prior to administering the vaccine composition 311 to the non-human animal 304 through a needle 308, or the first and second feeds 301,302 may be kept separated and for such an embodiment, the injector 303 may then comprise two or more needles 308 or a single needle 308 may comprise a dual or multiple lumen.

The administering of the tag nucleic acid molecule 100,200 may take place concurrently with the vaccine composition 311, in the sense that the tag nucleic acid molecule 100,200 and the vaccine composition 311 may both be administered in the course of a single process, such as a single combined vaccination and tagging process, or a single injection cycle of the vaccine composition 311. For example, the injection apparatus 300 may be configured such that administering the tag nucleic acid molecule 100,200 may take place within 20 minutes of administering the vaccine composition 311 (which may be administered before or after the tag nucleic acid molecule 100,200), preferably within 10, 5, 3 or 1 minute(s) of administering the vaccine composition 311.

In preferred embodiments the tag nucleic acid molecule 100,200 and the vaccine composition 311 are administered simultaneously, or at least during the same injection cycle, i.e. during the same period that the non-human animal 304 is being restrained in order to administer the vaccine composition 311. In this way the non-human animal 304 is restrained for a minimum amount of time and the administration of the tag nucleic acid molecule 100,200 does not unduly slow the administration of the vaccine composition 311.

However, it is also envisaged that a tag nucleic acid molecule 100,200 may be administered before and/or after the administering of the vaccine composition 311, as the non-human animal 304 passes through or past the injection apparatus 300. For example, a non-human animal 304, such as a fish, may pass along a conveyance system 309, and in so doing, may pass through a first injection station 310 where one of a tag nucleic acid molecule 100,200 or a vaccine composition 311 is administered, and then pass through a second injection station (not shown) where the other of the tag nucleic acid molecule 100,200 or vaccine composition 311 is administered.

Preferably, the non-human animal 304 passes along a conveyance system 309 and in doing so passes through a single injection station 310 where the tag nucleic acid molecule 100,200 and the vaccine composition 311 are both administered, either in a single injection or via multiple injections.

In more preferred embodiments, the injection apparatus 300 may be configured to administer the tag nucleic acid molecule 100,200 and the vaccine composition 311 to a same point of injection on the non-human animal 304. In this way it may be possible to reduce potential for damage to the non-human animal 304 by avoiding the need to create multiple points of injection. The tag nucleic acid molecule 100,200 may be administered through the same needle 308 as the vaccine composition 311. For example, injection apparatus 300 may be configured to administer the tag nucleic acid molecule 100,200 together with the vaccine composition 311 in a mixed solution, e.g., at the point of delivery to the non-human animal 304, or configured to administer the tag nucleic acid molecule 100,200 via a first lumen of the needle 308 together with the vaccine composition 311 via a second lumen of the needle 308, allowing the tag nucleic acid molecule 100,200 and the vaccine composition 311 to be kept separated. Alternatively, the injection apparatus 300 may be configured to administer the tag nucleic acid molecule 100,200 through the same needle 308 as the vaccine composition 311 at the same injection site but at a sequential point in time, for example, before or after a dose of vaccine composition 311 has been administered to the non-human animal 304. Similarly, the injection apparatus 300 may be configured to administer the tag nucleic acid molecule 100,200 and the vaccine composition 311 through different needles 308, either simultaneously or sequentially.

Administering the tag nucleic acid molecule 100,200 and vaccine composition 311 to a same point of injection on the non-human animal 304 may also include injection to a localised area of the non-human animal 304 through two needles 308 which are in close proximity, for example, within a separation of less than 50 mm and preferably less than 40, 30, 20, 10, 5 or 3 mm.

In some instances, for example, where it might benefit the configuration of the injection apparatus 300, administration of the tag nucleic acid molecule 100,200 and the vaccine composition 311 may occur at different points of injection on the non-human animal 304, e.g., through separate injectors 307 or separate needles 308 of the injection apparatus 300.

While the injection apparatus 300 in FIG. 1 is shown with a single needle 308, the injection apparatus may comprise multiple needles 308 to deliver one or both of the tag nucleic acid molecule 100,200 and the vaccine composition 311, for example, as an array of micro-needles, where that is desired.

In the embodiments herein involving the use of a vaccine composition 311, the vaccine composition 311 and the tag nucleic acid molecule 100,200 are provided for separate, sequential or simultaneous administration to the non-human animal. In other words, the tag nucleic acid molecule 100,200 is for use together with, or in combination with, the vaccine composition 311.

In some embodiments, the vaccine composition 311 and the tag nucleic acid molecule 100,200 may be administered in the form of an admixture, i.e. the tag nucleic acid molecule and the vaccine composition may be pre-mixed prior to administration to form an admixture comprising both a vaccine composition 311 and a tag nucleic acid molecule 100,200, which is later administered to the non-human animal 304. Alternatively, the vaccine composition 311 and the tag nucleic acid molecule 100,200 may be provided, or kept, separately and may be mixed either just before or during the administration process, for example, by administering the vaccine composition 311 and tag nucleic acid molecule 100,200 together in a single injection using a single needle 308 operably connected to a volume of the vaccine composition 311 and a separate volume of the tag nucleic acid molecule 100,200.

As used herein, “concurrent administration” includes the administration of an admixture comprising the tag nucleic acid molecule 100,200 and the vaccine composition 311, but also the administration of one component followed by the administration of the other component, i.e. the two components do not need to be administered at exactly the same time for the administration to be considered concurrent. Alternatively put, if the two components are administered in the course of a single process comprising the administration of one component followed by the administration of the other component, then this may be considered to be concurrent administration. Accordingly, in some embodiments, concurrent administration may include administration protocol wherein the tag nucleic acid molecule 100,200 and the vaccine composition 311 are administered within 20 minutes of each other e.g. within 15, 10, 5, 4, 3 or 2 minutes of each other.

As used herein, “simultaneous administration” refers includes the administration of the tag nucleic acid molecule 100,200 and the vaccine composition 311 at the same time (e.g. in the form of an admixture) or at substantially the same time as each other. Again, the two components do not need to be administered at exactly the same time for the administration to be considered simultaneous. If the two components are administered within 2 minutes of each other, for example within 1 minute, or 30 seconds or 10 seconds of each other, then this may be considered to be simultaneous administration. Simultaneous administration may involve administration of both components together by one administration (e.g. one injection) or two separate administrations (e.g. two separate injections), at the same time, or one after the other. In one embodiment the tag nucleic acid molecule 100,200 and the vaccine composition 311 may be provided in pre-mixed form. In another embodiment the tag nucleic acid molecule 100,200 and the vaccine composition 311 may be provided separately and may be pre-mixed by the user, for example just prior to use (i.e. just prior to administration), or during use. For example, in an administration device (e.g. injector 303), feeds 301 or reservoirs containing the tag nucleic acid molecule 100,200 and the vaccine composition 311 may be mixed or combined together within the device, prior to administration to the non-human animal 304.

The tag nucleic acid molecule 100,200 and the vaccine composition 311 may be provided separately within a combination product for such use. For example, in one embodiment the product may be configured to allow the two components to be pre-mixed prior to use. In another embodiment, the product may be configured for the components to be administered separately. In a further embodiment, the product may be configured to allow the two components to be combined or mixed in use, for example within an injector device.

In some embodiments, it may be desirable to avoid mixing the tag nucleic acid molecule 100,200 with the vaccine composition 311 significantly before injection in order to avoid creating any regulatory complications with regard to the conditions associated with the regulatory approval of the vaccine for use. However, depending on these imposed conditions of any regulatory approval for the use of the vaccine composition 311, it may be an option to mix the tag nucleic acid molecule 100,200 with the vaccine composition 311 prior to supplying the vaccine composition 311 to the injection apparatus 300. For example, the vaccine composition 311 may be packaged in a way to facilitate such mixing of the tag nucleic acid molecule 100,200 with the vaccine composition 311, e.g., on site in a location where the non-human animal 304 is due to be vaccinated shortly before the vaccine composition 311 is administered, as is discussed further below.

In this regard, provided herein is a pharmaceutical package 411, which may be in the form of a bag or other sealed container, comprising a first compartment 412 containing a vaccine composition 311 for a non-human animal 304 and a second compartment 413 containing a tag nucleic acid molecule 100,200, wherein the pharmaceutical package 411 is arranged to allow mixing of the tag nucleic acid molecule 100,200 with the vaccine composition 311, e.g., on site in a location where the non-human animal 304 is due to be vaccinated, prior to administering the vaccine composition 311 to the non-human animal 304. This mixing may be achieved through rupture of one of the compartments to allow the tag nucleic acid molecule 100,200 to infiltrate the vaccine composition 311. In order to facilitate this rupture, the compartment may comprise a frangible or tearable wall 414 separating the tag nucleic acid molecule 100,200 from the vaccine composition 311. In another embodiment, this mixing may be achieved through opening of a closure sealing off one or both of the first and/or second compartments 412,413. The mixing may be conducted by the end user of the pharmaceutical package 411 shortly before the tag nucleic acid molecule 100,200 and vaccine composition 311 are administered to the non-human animal 304. The pharmaceutical package 411 may further comprise an outlet 415 which can be operably connected to an injector 303 in order for the vaccine composition 311 and the tag nucleic acid molecule 100,200 to be administered to the non-human animal 304.

The tag nucleic acid molecule 100,200 may be present in the pharmaceutical package 411 in the form of a tag nucleic acid molecule product, for example, in a solution of such tag nucleic acid molecules 100,200 comprising suitable buffers and additives as defined above, e.g., as a nucleic acid tracer product, which can be combined easily with the vaccine composition 311 (e.g., through being soluble in the vaccine composition) to form a single admixture that can be administered to the non-human animal.

In addition, provided herein is a system for administering a vaccine-tag product to a non-human animal, the system comprising injection apparatus 300 and a supply of vaccine-tag product 411 for administering to the non-human animal 304. In this embodiment, the vaccine-tag product is a combination product comprising:
(a) a vaccine composition 311 for vaccination of a non-human animal 304; and
(b) a tag nucleic acid molecule 100,200 which
   (i) comprises an ID sequence which is unique to the tag and may be distinguished from the ID sequences of other tag molecules, and
   (ii) is detectable in or on said non-human animal 304, or in a body tissue or fluid sample from the non-human animal 304 to which it has been administered.

In another embodiment, the vaccine composition 311 and the tag nucleic acid molecule 100,200 are administered separately, that is as separate compositions or formulations. In other words, they are not mixed before the administration process. In such embodiments the vaccine composition 311 and tag nucleic acid molecule 100,200 may be provided, or formulated, for administration by the same or different administration routes. In some embodiments, the vaccine composition 311 and tag nucleic acid molecule 100,200 may be kept separately and administered separately. This may include concurrent or sequential administration, for example, at spaced apart time intervals. In some embodiments, this may be less preferred, in order to minimise handling of the animals and/or to reduce the number of separate administrations. Separate administration may be done, for example, using two separate injections, i.e. using two needles, or using a single injection with a dual bore needle.

As noted above, it is desirable as far as is possible to avoid causing damage to the animal e.g. bruising or lacerations, when the tag nucleic acid molecule 100,200 is administered. Accordingly, where the tag nucleic acid molecule 100,200 and a vaccine composition 311 are administered separately, e.g. by separate injections or infusions or such like, the sites of administration (e.g. the injection sites) may be selected to be in close proximity to each other. In some embodiments, the two injection sites may be within 50 mm of each other, such as within 40, 30, 20, 10 or 5 mm of each other. In some embodiments, the two injections may be administered to the same site.

Once the tag nucleic acid molecule 100,200 has been administered to the non-human animal 304, it persists stably in the animal 304 so that it can be detected at a point in the future. Generally speaking, the tag nucleic acid molecule 100,200 is administered in such a manner that it cannot enter the germ cells of the animal, and thus cannot be passed on to any offspring that the animal 304 may have. Accordingly, in an embodiment the tag nucleic acid molecule 100,200 is not administered to the sex organs of an animal 304, or at a site from which it can reach the sex organs, or the germ cells. As noted above, the tag nucleic acid molecule 100,200 may be non-coding and/or may comprise a stop codon at or near the 5′ end. Accordingly, even if the tag nucleic acid molecule 100,200 is integrated into the genome of the host cells, it will not be transcribed.

The tag nucleic acid molecule 100,200 may be detected in or on the body of an animal 304 to which it has been administered. In an embodiment it is detectable in a body tissue or fluid sample taken from the animal 304 to which it has been administered. It will be understood that the nature of the sample in which the tag nucleic acid molecule 100,200 is detectable may depend on the method by which it was administered. The administration may be localised such that the tag nucleic acid molecule 100,200 largely remains at or close to the site of administration. Thus, the tissue or fluid at the site of administration may be sampled. For example, if the tag nucleic acid molecule 100,200 was administered via injection into the abdominal cavity, it may be detected from a sample taken using a swab of the abdominal cavity.

The sample may be any body tissue or fluid sample derived from the animal 304. For example, the sample may be a sample of muscle, skin, blood or a blood-derived product such as plasma or serum, peritoneal fluid, interstitial fluid, lymph, or pleural fluid, or a swab or wash from the site of administration. Methods and procedures for sample taking are well known in the art, and include biopsy, aspiration, swabs and such like. In embodiments where the tag nucleic acid molecule 100,200 is attached to a solid carrier, the sample taken from the animal 304 may comprise part or all of the carrier, or may be taken from the immediate vicinity of the carrier (i.e. from the tissue to which the carrier was administered, and the surrounding area). In some embodiments the carrier may be removed or detached from the animal 304 and used for the detection. Where there is a solid carrier, the tag nucleic acid molecule may be separated from the carrier prior to carrying out the detection.

The tag nucleic acid molecule 100,200 may be detected by any suitable means known in the art. In order to facilitate the detection process, the tag nucleic acid molecule 100,200 in the sample may be amplified. This amplification step increases the number of copies of the tag nucleic acid molecule 100,200 which are present in the sample, and thus increases the strength of the signal which can be detected. A variety of methods for amplification of nucleic acids are known and any of these may be used, including isothermal and thermal cycling methods, for example PCR, and derivatives thereof, LAMP, NASBA, LCR, 3SR, and RCA.

Amplification methods typically involve the use of one or more primers, as discussed above. Conveniently, the amplification may be done in the form of a PCR reaction involving primers which are capable of binding to the primer binding sites on the tag nucleic acid molecule 100,200. The tag nucleic acid molecule 100,200 may be detected by detecting the presence of a successful amplification reaction, such as a PCR reaction, i.e. by detecting an amplicon or a PCR product.

In some embodiments, the ID sequence of the tag nucleic acid molecule 100,200 may be contained partially or fully within a primer binding sequence, i.e. a given tag nucleic acid molecule 100,200 may be distinguishable from other tag nucleic acid molecules 100,200 by virtue of its primer binding sequences. The tag nucleic acid molecule 100,200 may therefore be detected using specific primers which are capable of binding to the primer binding sites for that tag nucleic acid molecule 100,200. In some embodiments, the primers may comprise a detectable label, such that the binding of the primers to the tag nucleic acid molecule 100,200 can be detected, and therefore the presence and identity of the tag nucleic acid molecule 100,200 can be confirmed. In this case, the primers can act as detection probes which are capable of indicating the presence and identity of the tag nucleic acid molecule 100,200, and thus the use of a separate detection probe is not required.

Alternatively, the ID sequence may be between the primer binding sequences, and may be capable of hybridising to a detection probe. The tag nucleic acid molecule 100,200 may thus be detected using a specific detection probe capable of binding to the ID sequence for that tag nucleic acid molecule 100,200. The detection probe may thus be viewed as a hybridisation probe which is capable of being detected. Various types of hybridisation probe are known in the art, including simple single-stranded oligonucleotides capable of hybridising to a target sequence, here the ID sequence, molecular beacons, scorpion probes, padlock probes, and invader probes etc., and any such suitable probe may be used. Thus, multi-part probes and probe systems may be used, for example to obtain further signal amplification. In some embodiments, the detection probe, or a part of a detection probe system, may comprise a detectable label, such that the binding of the detection probe to the tag nucleic acid molecule 100,200 can be detected, and therefore the presence and identity of the tag nucleic acid molecule 100,200 can be confirmed.

The detectable label which is used in either the primers or the detection probe or probe system may be any label which is capable of providing a signal that can be detected, for example fluorescent molecules (e.g. fluorescent proteins or organic fluorophores), colorimetric moieties (e.g. coloured molecules or nanoparticles), particles, for example gold or silver particles, quantum dots, radioisotopes, chemiluminescent molecules, and the like. In particular, any spectrophotometrically or optically-detectable label may be used. The detectable label may be distinguishable by colour, but any other parameter may be used e.g. size.

In a particular embodiment, the detectable label comprises a fluorescent molecule, e.g. a fluorophore. Fluorescent molecules that may be used to label nucleotides are well known in the art. Exemplary fluorophores include ATTO425, Cy3, Cy5, Cy7, and AF488, though any suitable fluorophores may be used. Fluorophores have been identified with excitation and emission spectra ranging from UV to near IR wavelengths. Thus, the fluorophore may have an excitation and/or emission wavelength in the UV, visible or IR spectral range. The fluorophore may be a peptide, small organic compound, synthetic oligomer or synthetic polymer. In some embodiments, the fluorophore is a small organic compound.

In some embodiments, the tag nucleic acid molecule 100,200 is detected using a real time PCR reaction. Such reactions are well known in the art, and may be conducted using a labelled detection probe, or using labelled primers. For example, the tag nucleic acid molecule 100,200 may be detected via real time PCR using a detection probe which is capable of binding to the tag nucleic acid molecule 100,200 and which comprises a fluorophore and a quencher. In the absence of the tag nucleic acid molecule 100,200, the fluorophore on the detection probe is quenched by the quencher. During the annealing phase of the PCR reaction, the detection probe (together with the primers) can hybridise to the tag nucleic acid molecule 100,200, before polymerisation is then initiated from the primers. When the polymerase reaches the detection probe, the detection probe is degraded and the fluorophore is separated from the quencher, resulting in a detectable fluorescent signal. The strength of this fluorescent signal can be monitored in real time throughout the PCR reaction to measure the amplification of the PCR product, and therefore to indicate the presence of the tag nucleic acid molecule 100,200.

It will be understood that in situations where it is desired to distinguish between multiple different tag nucleic acid molecules 100,200 (i.e. between different species of tag nucleic acid molecule 100,200), each tag nucleic acid molecule 100,200 will be assigned a different detectable label. The primers and/or detection probe specific for each tag nucleic acid molecule 100,200 will comprise the specific detectable label assigned to that tag nucleic acid molecule 100,200, such that the tag nucleic acid molecules 100,200 can be distinguished from each other.

In embodiments where it is necessary to distinguish between large numbers of tag nucleic acid molecules 100,200, it may not be possible to assign a different detectable label to each tag nucleic acid molecule 100,200 (i.e. the number of tag nucleic acid molecules 100,200 to be distinguished may be larger than the number of different detectable labels which are available). Various techniques are known in the art for increasing labelling capacity, including for example ratio-labelling or sequential labelling strategies. In one embodiment, the tag nucleic acid molecules 100,200 may be detected using a barcoding system, whereby each tag nucleic acid molecule 100,200 is assigned a barcode comprised of a sequence of two or more detectable labels. The tag nucleic acid molecules 100,200 may then be detected by sequentially hybridising and detecting a set of detection probes having the detectable labels present in the barcode. In this regard, the multiplicity of the detection method may be exponentially increased by simply increasing the length of the barcode sequences. Such barcoding systems are well known in the art, and the skilled person would be capable of apply any such suitable system to the present methods.

In other embodiments, the tag nucleic acid molecule 100,200 may be detected by methods which involve or comprise sequencing. The tag nucleic acid molecules 100,200, or more particularly amplicons thereof, may be prepared for and subjected to sequencing by any desired sequencing protocol or method, including high throughput or next generation sequencing methods. In an embodiment, tag nucleic acid molecules 100,200, particularly those that comprise ID sequences in the form of barcodes may be detected by sequencing-by-hybridisation methods, including those that use sequential hybridisation reactions with read-out probes (which may be seen as a form of detection probe) to decode the barcode.

Other detection modalities are known in the art, including for example, distinguishing the amplicons of the ID sequence by size. Any convenient method may be used.

Further, the amplification and/or detection procedures may be carried out homogenously, or in a so-called “in-solution” or liquid phase format, or heterogeneously, using a solid phase. Using a solid phase, including particularly a particulate solid phase, may assist in the separation of amplicons, or products labelled with detection probes etc., and/or may allow for washing steps etc. to be included. Solid phase-based methods may thus be convenient. For example, one more amplification primers, and/or detection probes etc. may be provided in immobilised form, linked to a solid support, or with means for immobilisation to a solid support, such as for example a member of an affinity binding pair (e.g. antibody-hapten pair) which is able to bind to its cognate affinity binding partner provided on the solid support. Magnetic particles, particularly superparamagnetic particles are suitable for use as such a solid support.

The tag nucleic acid molecules 100,200 described herein may be used in a number of different applications to provide information about the non-human animals 304 to which they are administered, and to allow those animals 304 to be identified and traced. It will be understood that the system in which the present tag nucleic acid molecules 100,200 are used may be set up before the tag nucleic acid molecules 100,200 are administered to the animals 304 to be tagged, i.e. the information that is to be indicated by each tag nucleic acid molecule 100,200 may be decided upon and recorded in advance. The present tag nucleic acid molecules 100,200 may be used to provide information about and to distinguish between individual animals 304 or groups of animals 304 of any appropriate size based on any desired characteristic.

Methods of distinguishing between individual animals 304 are particularly useful where the animals 304 have significant value individually. For example, the present tag nucleic acid molecules 100,200 may be used to distinguish between individual animals 304 which have significant commercial value either as food, such as tuna, or otherwise, such as aquarium fish, or other domestic or sports animals, or breeding animals and such like etc.

As noted above, the present methods and tag nucleic acid molecules 100,200 may be applied to farmed animals 304. In this regard, the present tag nucleic acid molecules 100,200 may be used to distinguish between different farms and/or different groups of animals 304 within a single farm. In some embodiments, the non-human animals 304 to which the tag nucleic acid molecules 100,200 are administered may be farmed fish. The tag nucleic acid molecules 100,200 may therefore be used to distinguish between groups of fish within a given fish farm. For example, the present tag nucleic acid molecules 100,200 may be used to indicate the pond of a given fish (i.e. by assigning a different tag nucleic acid molecule to each pond, and administering the assigned tag nucleic acid molecule to each fish within that pond). An equivalent approach may be used to distinguish between any other desired characteristic of the fish within a given farm, such as the sub-type, breed, or batch of fish, or indeed in an analogous fashion between any groups of farmed animals. In a particular embodiment, the non-human animals 304 are farmed salmon, and thus the present tag nucleic acid molecules 100,200 may be used to distinguish between salmon of different ponds, sub-types, breeds or batches on a given fish farm. In an embodiment, the tags 100,200 may be used to designate production batches, or time or date of production, e.g. as a type of “date-stamp”.

The present methods and tag nucleic acid molecules 100,200 may be similarly used on a larger scale to distinguish between animals 304 from different sources of origin, e.g. from different farms and/or from different locations. This may be particularly useful in the context of fish farming, where the same species of fish can be farmed in different locations and can have different values depending on where and how they were produced. For example, salmon farmed in Chile may be genetically identical to and otherwise indistinguishable from salmon farmed in Norway, but may have significantly different value to the Norwegian fish. It may therefore be desirable to be able to determine the origin and provenance of a given animal 304, and this may be done using the present methods and tag nucleic acid molecules 100,200. In some embodiments, the methods disclosed herein may be used to determine the country of origin of farmed salmon.

As outlined above, the present invention provides multiple embodiments whereby the tag nucleic acid molecules 100,200 are administered or used together with a vaccine composition 311. Accordingly, where the tag nucleic acid molecule 100,200 and the vaccine composition 311 are administered together (including both simultaneous administration and sequential administration as part of the same process), the tag nucleic acid molecule 100,200 may also be used as proof of vaccination, whereby the present of the tag 100,200 indicates that a vaccine was administered.

In the same way that different tag nucleic acid molecules 100,200 may provide information about the animals 304 to which they were administered, so the present tag nucleic acid molecules 100,200 may also be used to provide information about the vaccine composition 311 with which they were co-administered. In this regard, the tag nucleic acid molecules 100,200 may be used to indicate the type of vaccine or the specific batch of vaccine that was administered, the time of administration, or any other desired characteristic of the vaccine composition 311.

The invention will now be described in more detail in the following non-limiting drawings and Examples.

### Example 1

A first example DNA sequence 100 is provided in SEQ ID NO: 1. The DNA sequence 100 may be as follows:

[SEQ ID NO: 1]
tgaagtagct ccataatcag aagatgcgct accgtacaca
acggcggttt aaactttgta gacctcacgc ggctgctcag
gtcttgcgca toccatactt cacatggacg gaaagctgtg
ggtttgtatc gataagccgt aggttcctag gatcgagtca
cccaaacaac ttcgagagct cttctcacca accctttgaa
ctccgaaatg ccgtgaacac atccccttag ttggcattgg
tctactaacc gagtatcgta caaagcgaga caggtctggc
ccctactgct tggatgcgaa ctaacagtgt tcctatatcc
gtttatactg tggtcgattt cgaaccgata tctcaaagag
agcgccggca gagccttagt atcggaactg gcgagtgtgt
ttgggtgagg actattatgt ggttggagta gagcgaatct
CC

or an identity of 80% or more thereto, for example 85%, 90%, 95%, 96%, 97%, 98% or 99% or more.

The group of nucleotides highlighted in bold in the example sequence are the binding sites for the forward and reverse primers respectively; and the central group of nucleotides highlighted in bold and italics is the binding site for the detection probe; and the 2× four base pair segments at the 5′ and 3′ ends are the nuclease precautions. The forward and reverse primers may have sequences as set out in SEQ ID NOs: 2 and 3, respectively.

[SEQ ID NO: 2]
gtagctccat aatcagaaga tgcg
[SEQ ID NO: 3]
attcgctcta ctccaaccac ataatag

The detection probe may have a sequence as set out in SEQ ID NO: 4.

[SEQ ID NO: 4]
tccgatacta aggctctgcc ggcgct

In this example, the forward primer was selected to be between 21 and 25 base pairs long, for example 24 base pairs.

Preferably the binding site for the forward primer comprises nucleotides 5 to 28 of SEQ ID NO: 1, i.e. gtagctccat aatcagaaga tgcg (SEQ ID NO: 5), or a sequence with greater than 95% identity thereto.

The reverse primer was selected to be between 22 and 28 base pairs long, for example 27 base pairs.

Preferably the binding site for the reverse primer comprises nucleotides 412 to 438 of SEQ ID NO: 1, i.e. cta ttatgtggtt ggagtagagc gaat (SEQ ID NO: 6), or a sequence with greater than 95% identity thereto.

The binding site for the detection probe may be between 16 and 30 base pairs long, for example 26 base pairs.

Preferably the binding site for the detection probe comprises nucleotides 361 to 386 of SEQ ID NO: 1, i.e. agcg ccggcagagc cttagtatcg ga (SEQ ID NO: 7), or a sequence with greater than 95% identity thereto.

Preferably the sequence 100 includes an initial four base pair sequence of TGAA closest to the 5′ end of the tag sequence and preferably a final four base pair sequence of CTCC at the 3′ end of the tag sequence 100.

A complementary sequence could comprise:

[SEQ ID NO: 8]
acttcatcga ggtattagtc ttctacgcga tggcatgtgt
tgccgccaaa tttgaaacat ctggagtgcg ccgacgagtc
cagaacgcgt agggtatgaa gtgtacctgc ctttcgacac
ccaaacatag ctattcggca tccaaggatc ctagctcagt
gggtttgttg aagctctcga gaagagtggt tgggaaactt
gaggctttac ggcacttgtg taggggaatc aaccgtaacc
agatgattgg ctcatagcat gtttcgctct gtccagaccg
gggatgacga acctacgctt gattgtcaca aggatatagg
caaatatgac accagctaaa gcttggctat agagtttctc
tcgcggccgt ctcggaatca tagccttgac cgctcacaca
aacccactcc tgataataca ccaacctcat ctcgcttaga
gg

### Example 2

A further example DNA sequence 100 is provided below. This is a double stranded DNA sequence, having a first strand with the sequence from 5′ to 3′ as set out in SEQ ID NO: 9 and a second strand with the complementary sequence from 3′ to 5′ as set out in SEQ ID NO: 10.

[SEQ ID NO: 9]
TAG TTC ACG GGC AAT GTT CGG CCG
TTA ATT GTT AAC TTA ATT GTT AAC
ACG GGC AAT ACG GG
[SEQ ID NO: 10]
ATC AAG TGC CCG TTA CAA GCC GGC
AAT TAA GAA TTG AAT TAA CAA
TTG TGC CCG TTA TGC CC

The DNA tag may have the sequence set out above or an identity of 80% or more thereto, for example 85%, 90%, 95%, 96%, 97%, 98% or 99% or more. The group of nucleotides highlighted in bold in the example sequences are the binding sites for the forward and reverse primers respectively.

The forward and reverse primers may have sequences as set out in SEQ ID NOs: 11 and 12, respectively (both written from 5′ to 3′).

[SEQ ID NO: 11]
TAG TTC ACG GGC AAT GTT CGG
[SEQ ID NO: 12]
CC CGT ATT GCC CGT GTT AAC

In this example, the forward primer was selected to be between 18 and 24 base pairs long, for example 21 base pairs.

Preferably the binding site for the forward primer comprises nucleotides 1 to 21 of SEQ ID NO: 10 i.e. ATC AAG TGC CCG TTA CAA GCC (in 3′ to 5′ direction) (SEQ ID NO: 13), or a sequence with greater than 95% identity thereto.

The reverse primer was selected to be between 18 and 24 base pairs long, for example 20 base pairs.

Preferably the binding site for the reverse primer comprises nucleotides 43 to 62 of SEQ ID NO: 9, i.e. GTT AAC ACG GGC AAT ACG GG (SEQ ID NO: 14), or a sequence with greater than 95% identity thereto.

1. A method of tagging a non-human animal, said method comprising administering to said animal a tag nucleic acid molecule, wherein the tag nucleic acid molecule:
(i) comprises an ID sequence which is unique to the tag, which is non-coding and/or cannot be transcribed, and which may be distinguished from the ID sequences of other tag molecules, and
(ii) is detectable in or on said animal, or in a body tissue or fluid sample from said animal.

2. A method of identifying a non-human animal, said method comprising detecting in or on said animal, or in a body tissue or fluid sample from said animal the presence and identity of a tag nucleic acid molecule, wherein the tag nucleic acid molecule has previously been administered to said animal, and comprises an ID sequence which is unique to the tag which is non-coding and/or cannot be transcribed, and which may be distinguished from the ID sequences of other tag molecules.

3. The method of claim 1 or 2, wherein the animal is a farmed animal.

4. The method of any one of claims 1 to 3, wherein the animal is a non-mammalian aquatic animal.

5. The method of any one of claims 1 to 4, wherein the animal is a fish, preferably wherein the fish is salmon, tuna, cod or sea trout.

6. The method of any one of claims 1 to 5, wherein the same tag nucleic acid molecule is administered to a cohort or population of animals.

7. The method of any one of claims 1 to 6, wherein the tag nucleic acid molecule is DNA.

8. The method of any one of claims 1 to 7, wherein the tag nucleic acid molecule further comprises a nucleotide sequence encoding a vaccine component.

9. The method of claim 8, wherein tag nucleic acid comprises one or more stop codons at or near the 5′ end.

10. The method of any one of claims 1 to 9, wherein the tag nucleic acid molecule is at least 36 nucleotides in length and/or no more than 1000 nucleotides in length.

11. The method of any one of claims 1 to 10, wherein nuclease resistance of the tag nucleic acid molecule is increased by one or more of the following:
(i) a coiled or supercoiled structure;
(ii) a circular configuration;
(iii) a plasmid or plasmid-like structure;
(iv) one or more modifications which increase nuclease resistance, optionally wherein said modifications are selected from phosphorothioate linkages and/or modified nuclease-resistant nucleotide or nucleoside residues and/or PNA or LNA residues.
(v) the absence of a restriction endonuclease recognition and/or cleavage site.

12. The method of any one of claims 1 to 11, wherein the tag nucleic acid molecule comprises primer binding sites for amplification of the ID sequence.

13. The method of claim 12, wherein the primer binding sites:
(i) are for binding of a forward and a reverse primer; and/or
(ii) flank the ID sequence; and/or
(iii) are 16-25 nucleotides in length; and/or
(iv) are separated from one another by at least 6 nucleotides;
and optionally wherein the tag nucleic acid molecule additionally comprises a probe binding site sequence between the primer binding sites.

14. The method of any one of claims 1 to 13, wherein the tag nucleic acid molecule has a melting temperature of 70° C. or more and/or a GC content of greater than 60%.

15. The method of any one of claims 1 to 14, wherein the tag nucleic acid molecule is detectable or detected by a PCR reaction.

16. The method of claim 15, wherein the PCR primers are labelled, and the PCR product is detected by detecting the label(s), or wherein the PCR product is detected by detecting the hybridisation of a probe to the probe binding site in the product.

17. The method of any one of claims 1 to 16, wherein the tag nucleic acid molecule is administered by injection.

18. The method of claim 17, wherein the animal is a fish and the tag nucleic acid molecule is administered by injection into the abdominal cavity.

19. The method of any one of claims 1 to 18, wherein the tag nucleic acid molecule is provided on or in a solid carrier which is inserted into or attached to the non-human animal.

20. The method of any one of claims 1 to 19, wherein the sample is a sample of muscle, skin, blood, plasma, serum, peritoneal fluid, interstitial fluid, lymph, or pleural fluid or a swab or wash taken from a body site of the animal.

21. A combination product comprising:
(a) a vaccine composition for vaccination of a non-human animal; and
(b) a tag nucleic acid molecule which
(i) comprises an ID sequence which is unique to the tag, which is non-coding and/or cannot be transcribed, and which may be distinguished from the ID sequences of other tag molecules, and
(ii) is detectable in or on said animal, or in a body tissue or fluid sample from an animal to which it has been administered.

22. The combination product of claim 21, wherein the tag nucleic molecule is as defined in any one of claims 7 to 15.

23. The combination product of claim 21 or 22, wherein the combination product is for use in vaccinating a non-human animal, and said vaccine composition and tag nucleic acid molecule are provided for separate, sequential or simultaneous administration to said animal.

24. The combination product for use according to claim 23, wherein the non-human animal is as defined in any one of claims 3 to 5.

25. The combination product for use according to claim 23 or 24, wherein the vaccine composition and the tag nucleic acid molecule are administered by injection.

26. A method of vaccinating and tagging a non-human animal, said method comprising administering to said animal (a) a vaccine composition together with a tag nucleic acid molecule or (b) a tag nucleic acid molecule which additionally comprises a nucleotide sequence encoding a vaccine component, wherein the tag nucleic acid molecule:
(i) comprises an ID sequence which is unique to the tag, which is non-coding and/or cannot be transcribed, and which may be distinguished from the ID sequences of other tag molecules; and
(ii) is detectable in or on said animal, or in a body tissue or fluid sample from said animal;
and wherein in (a) the vaccine composition and tag nucleic acid molecule are administered separately or in admixture.

27. A vaccine-tag nucleic acid molecule for use in vaccinating and tagging a non-human animal, wherein the vaccine-tag nucleic acid molecule comprises:
(a) a first nucleotide sequence which comprises an ID sequence which is unique to the tag, which is non-coding and/or cannot be transcribed, and which may be distinguished from the ID sequences of other tag molecules; and
(b) a second nucleotide sequence which encodes a vaccine component for vaccination of a non-human animal;
and wherein the vaccine-tag nucleic acid molecule is detectable in or on said animal.

28. Use of:
(a) a vaccine composition; and
(b) a tag nucleic acid molecule;
for the preparation of a combined product for use in vaccinating a non-human animal, wherein the tag nucleic acid molecule:
(i) comprises an ID sequence which is unique to the tag, which is non-coding and/or cannot be transcribed, and may be distinguished from the ID sequences of other tag molecules; and
(ii) is detectable in or on said animal, or in a body tissue or fluid sample from an animal to which it has been administered.

29. Use of a vaccine-tag nucleic acid molecule for the preparation of a vaccine-tag composition for use in vaccinating and tagging a non-human animal, wherein the vaccine-tag nucleic acid molecule comprises:
(a) a first nucleotide sequence which comprises an ID sequence which is unique to the tag, which is non-coding and/or cannot be transcribed, and which may be distinguished from the ID sequences of other tag molecules; and
(b) a second nucleotide sequence which encodes a vaccine component for vaccination of a non-human animal;
and wherein the vaccine-tag nucleic acid molecule is detectable in or on said animal, or in a body tissue or fluid sample from an animal to which it has been administered.

30. A vaccine injection apparatus for administering a tag nucleic acid molecule with a vaccine composition to a non-human animal, wherein the injection apparatus comprises:
(a) a first feed of a vaccine composition; and
(b) a second feed of a tag nucleic acid molecule;
wherein the injection apparatus is configured to administer the tag nucleic acid molecule when administering the vaccine composition to the non-human animal.

31. The apparatus of claim 30, wherein:
(i) a primary input port is provided on an injector of the injection apparatus to connect with the first feed of vaccine composition; and
(ii) a secondary input port is provided on the injector to connect with the second feed of tag nucleic acid molecule.

32. The apparatus of claim 30 or 31, wherein:
(a) the injector comprises a mixing chamber to mix the tag nucleic acid molecule with the vaccine composition prior to administering the vaccine composition; or
(b) the first and second feeds are kept separated.

33. The apparatus of any one of claims 30 to 32, wherein the injection apparatus is configured to administer the tag nucleic acid molecule and the vaccine composition to a same point of injection on the non-human animal.

34. The apparatus of any one of claims 30 to 33, wherein the injection apparatus is:
(a) configured to administer the tag nucleic acid molecule in admixture with the vaccine composition in a mixed solution; or
(b) configured to administer the tag nucleic acid molecule simultaneously with the vaccine composition via a second lumen of the needle; or
(c) configured to administer the tag nucleic acid molecule through the same needle as the vaccine composition at a sequential point in time.

35. A pharmaceutical package comprising:
(a) a first compartment containing a vaccine composition for a non-human animal; and
(b) a second compartment containing a tag nucleic acid molecule;
wherein the pharmaceutical package is arranged to allow mixing of the tag nucleic acid molecule with the vaccine composition prior to administering the vaccine composition to the non-human animal.

36. A system for administering a vaccine-tag product to a non-human animal, the system comprising injection apparatus and a supply of vaccine-tag product for administering to the non-human animal, wherein the vaccine-tag product is a combination product comprising:
(a) a vaccine composition for vaccination of a non-human animal; and
(b) a tag nucleic acid molecule; which (b)
(i) comprises an ID sequence which is unique to the tag and may be distinguished from the ID sequences of other tag molecules; and
(ii) is detectable in or on said animal, or in a body tissue or fluid sample from said animal to which it has been administered,
wherein said ID sequence is non-coding and/or cannot be transcribed.
